(19) United States
(12) Patent Application Publication
(10) Pub. No.: US 2026/0283811 A1
(43) Pub. Date: Sep. 24, 2026

(54) OFFSET ADAPTERS, TRIAL IMPLANT SYSTEMS, AND IMPLANT SYSTEMS ALLOWING FOR SELECTABLE ECCENTRICITY

(71) Applicant: DEPUY IRELAND UNLIMITED COMPANY, Ringaskiddy County Cork (IE)

(72) Inventors: Brian C. HODOREK, Winona Lake, IN (US); Alec CUNNINGHAM, Fort Wayne, IN (US); Russ M. PARROTT, Winona Lake, IN (US); J. Michael WIATER, Beverly Hills, MI (US); Anand M. MURTHI, Baltimore, MD (US); Matthew J. SMITH, Columbia, MO (US); Derek J. CUFF, Venice, FL (US); Andrew JAWA, Cambridge, MA (US); Luke AUSTIN, Haddonfield, NJ (US); Matt J. PURDY, Winona Lake, IN (US)

(73) Assignee: DEPUY IRELAND UNLIMITED COMPANY, Ringaskiddy County Cork (IE)

(21) Appl. No.: 19/670,161

(22) Filed: May 7, 2026

Related U.S. Application Data
(62) Division of application No. 18/059,231, filed on Nov. 28, 2022.
Application No. 18/059,231 is a continuation of application No. PCT/US2021/034223, filed on May 26, 2021.
(60) Provisional application No. 63/030,379, filed on May 27, 2020.

(51) Int. Cl. A61F 2/40; A61F 2/46; A61F 2/30

(52) U.S. Cl. CPC A61F 2/4014; A61F 2/4684; A61F 2002/30224; A61F 2002/30354; A61F 2002/305; A61F 2002/30538; A61F 2002/30604; A61F 2002/30617; A61F 2002/4018; A61F 2002/4687

(57) ABSTRACT

The present disclosure is directed offset adapters, trial implant systems, shoulder implant systems, and methods for total shoulder replacement, wherein the eccentricity of a humeral head relative to a humeral anchor and resected proximal portion of a humerus is selectable by a surgeon. A humeral head implant system includes a humeral head, an adapter, and humeral anchor. An offset adapter is operable to connect the humeral head to a humeral anchor. The offset adapter includes a first tapered cylindrical portion having a first axis, a second tapered cylindrical portion having a second axis, and the first axis being eccentrically disposed relative to the second axis of the adapter. A trial humeral head system includes a trial humeral head, a pin guide, and a trial adapter.

### CROSS REFERENCE TO RELATED APPLICATION

This application is a divisional of U.S. Application No. 18/059,231, filed Nov. 28, 2022, which is a continuation of PCT Application No. PCT/US2021/034223, filed May 26, 2021, and entitled “Offset Adapters, Trial Implant Systems, and Implant Systems Allowing for Selectable Eccentricity,” which claims priority benefit under 35 U.S.C. §119(e) of U.S. Provisional Application No. 63/030,379, filed May 27, 2020, and entitled “Offset Adapters, Trial Implant Systems, and Implant Systems Allowing for Selectable Eccentricity,” which are incorporated herein by reference in their entireties.

### TECHNICAL FIELD

The present disclosure relates generally to orthopedic surgery, implants, and methods for replacing an articulation joint, for example, in the repair of a shoulder joint. More specifically, but not exclusively, the present disclosure relates to orthopedic surgery, humeral offset adapters, trial implant systems, implant systems, and methods for total shoulder replacement, wherein the eccentricity of a humeral head relative to a humeral stem/anchor and a resected proximal portion of a humerus is a selectable by a surgeon.

### BACKGROUND

In a typical anatomic total shoulder replacement, a damaged humeral head is replaced with a spherically-shaped metal head, and a damaged glenoid is replaced with a smooth plastic bearing surface. In a partial shoulder replacement, a damaged humeral head may only be replaced.

Conventional humeral implants include a humeral head having an articular surface and a humeral anchor attachable to a resected humerus. The humeral anchor includes a post or stem that is receivable in a cavity of the resected humerus and the intramedullary canal of the humerus. The post or stem of the humeral anchor is centrally disposed. A plurality of humeral heads having cavities with different eccentricities relative to the central axis are required to allow a surgeon to select the desired humeral head offset for a particular patient.

### SUMMARY

Shortcomings of the prior art are overcome and additional advantages are provided through the provision, in one embodiment, of a humeral head implant system having, for example, a humeral head, an offset adapter, and humeral anchor. The humeral head includes a first portion and a second portion. The first portion has an articular surface, and the first portion and the second portion define a central axis extending from the first portion to the second portion. The second portion has a surface with a tapered cavity therein, the cavity has a recess therein, and the cavity defines an axis offset and eccentrically disposed relative to the central axis of the humeral head. The offset adapter includes a first tapered cylindrical portion having a first axis, a second tapered cylindrical portion having a second axis, and the first axis being eccentrically disposed relative to the second axis of the offset adapter. A distal end of the first tapered cylindrical portion includes a protrusion offset from the first axis of the first tapered cylindrical portion of the offset adapter and from the second axis of the second tapered cylindrical portion. The humeral anchor has a tapered cavity defining a central axis. The first tapered cylindrical portion of the offset adapter is receivable in the tapered cavity of the humeral head with the protrusion receivable in the recess in the tapered cavity of the humeral head, and the second portion of the offset adapter receivable in the cavity of the humeral anchor.

In another embodiment, an offset adapter for operably connecting a humeral head to a humeral anchor includes, for example, a first tapered cylindrical portion having a first axis, a second tapered cylindrical portion having a second axis, the first axis being eccentrically disposed relative to the second axis of the offset adapter, and a distal end of the first tapered cylindrical portion comprising a protrusion having a third axis offset from the first axis of the first tapered cylindrical portion of the offset adapter and from the second axis of the second tapered cylindrical portion.

In another embodiment, a trial humeral head system includes, for example, a trial humeral head, a pin guide, and a trial offset adapter. The trial humeral head includes an outer hemispherical surface, an inner surface extending between a peripheral edge of the outer hemispherical surface, an aperture extending from the outer hemispherical surface to the inner surface, the outer hemispherical surface defining a central axis extending from the outer hemispherical surface to the inner surface and through the aperture, and the inner surface comprising an eccentric cavity extending around the aperture. The pin guide includes an elongated member having a first end, a second end, and a passageway therethrough from the first end to the second end. An outwardly-extending member is secured to the first end of the elongated member. The outwardly-extending member is releasably securable in the eccentric cavity of the trial humeral head with the elongated member extending through the aperture of the trial humeral head and with the passageway of the pin guide concentric with the central axis of the trial humeral head. The trial offset adapter includes a tapered cylindrical portion, an outwardly-extending member secured to the tapered cylindrical portion, a passageway extending from the first tapered cylindrical portion to the outwardly-extending member, and the outwardly-extending member releasably and adjustably securable in the eccentric cavity of the trial humeral head.

In another embodiment, a surgical method includes, for example, assembling a trial humeral head and a pin guide, positioning the trial humeral head and pin guide against a resected proximal humerus, installing a pin through the pin guide and into the resected humerus, removing the trial humeral head and pin guide from the resected humerus and the installed pin, using the pin to prepare a cavity in the resected humerus, removing the pin from the resected humerus, removing the pin guide from the trial humeral head, securing an trial offset adapter to the trial humeral head, selecting the degree of eccentricity of the trial offset adapter relative to the trial humeral head to adjust the position of the trial humeral head relative to the resected humerus, positioning the assembled trial offset adapter and trial humeral head over the resected humerus with a tapered cylindrical portion of the trial offset adapter disposed in a cavity of a humeral anchor disposed in the resected proximal humerus to confirm the position relative to the resected humerus, and selecting a humeral head and offset adapter based on the assembled trial humeral head and trial adapter.

### BRIEF DESCRIPTION OF THE DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a side elevational view, in part cross-section, of a humeral head implant system secured to a resected proximal portion of a humerus of a patient, according to an embodiment of the present disclosure;

FIG. 2 is an exploded, top perspective view of the humeral head implant system of FIG. 1, according to an embodiment of the present disclosure;

FIG. 3 is an exploded, bottom perspective view of the humeral head implant system of FIG. 1, according to an embodiment of the present disclosure;

FIG. 4 is a bottom perspective view of the humeral head of FIGS. 1-3, according to an embodiment of the present disclosure;

FIG. 5 is a side perspective view of the offset adapter of FIGS. 2 and 3, according to an embodiment of the present disclosure;

FIG. 6 is a perspective view of the offset adapter of FIG. 5, according to an embodiment of the present disclosure;

FIG. 7 is a top view of the offset adapter of FIG. 6, according to an embodiment of the present disclosure;

FIG. 8 is a bottom view of the offset adapter of FIG. 6, according to an embodiment of the present disclosure;

FIG. 9 is a bottom view of the assembled humeral head and offset adapter of FIGS. 2 and 3, according to an embodiment of the present disclosure;

FIG. 10 is a bottom view of the humeral head, offset adapter, and the humeral anchor of FIG. 1, according to an embodiment of the present disclosure;

FIG. 11 is a cross-sectional view of the humeral head, offset adapter, and the humeral anchor taken along line 11-11 in FIG. 10, according to an embodiment of the present disclosure;

FIG. 12 are perspective views of a plurality of different offset adapters, according to the embodiments of the present disclosure;

FIG. 13 are top views of the plurality of different offset adapters of FIG. 12, according to the embodiments of the present disclosure;

FIG. 14 are bottom views of the plurality of different offset adapters of FIG. 12 secured to a humeral head, according to the embodiments of the present disclosure;

FIG. 15 are perspective views of a trial humeral head system having a trial humeral head, a pin guide, and a trial offset adapter, according to the embodiments of the present disclosure;

FIG. 16 is a side elevational view, in part cross-section, of the trial humeral head and the pin guide secured to a resected proximal portion of a humerus of a patient, according to an embodiment of the present disclosure;

FIG. 17 is an exploded, top perspective view of the trial humeral head and the pin guide of FIG. 16, according to an embodiment of the present disclosure;

FIG. 18 is an exploded, bottom perspective view of the trial humeral head and the pin guide of FIG. 16, according to an embodiment of the present disclosure;

FIG. 19 is a top view of the trial humeral head of FIG. 16, according to an embodiment of the present disclosure;

FIG. 20 is a bottom view of the trial humeral head of FIG. 19, according to an embodiment of the present disclosure;

FIG. 21 is a partial cross-sectional perspective view of the trial humeral head taken along line 21-21 in FIG. 20, according to an embodiment of the present disclosure;

FIG. 22 is a top view of the pin guide of FIG. 16, according to an embodiment of the present disclosure;

FIG. 23 is a bottom view of the pin guide of FIG. 22, according to an embodiment of the present disclosure;

FIG. 24 is a bottom view of the assembled trial humeral head and pin guide of FIG. 16, according to an embodiment of the present disclosure;

FIG. 25 is a cross-sectional view of the assembled trial humeral head and pin guide taken along line 25-25 in FIG. 24, according to an embodiment of the present disclosure;

FIG. 26 is a partial cross-sectional side elevational view of the trial humeral head and the trial offset adapter of FIG. 15, disposed on a resected humerus and a humeral anchor, according to an embodiment of the present disclosure;

FIG. 27 is an exploded, top perspective view of the trial humeral head and the trial offset adapter of FIG. 26, according to an embodiment of the present disclosure;

FIG. 28 is an exploded, bottom perspective view of the trial humeral head and the trial offset adapter of FIG. 27, according to an embodiment of the present disclosure;

FIG. 29 is an enlarged, top perspective view of the trial offset adapter of FIG. 27, according to an embodiment of the present disclosure;

FIG. 30 is a bottom perspective view of the trial offset adapter of FIG. 29, according to an embodiment of the present disclosure;

FIG. 31 is a bottom view of the assembled trial humeral head and trial offset adapter of FIGS. 27 and 28, according to an embodiment of the present disclosure;

FIG. 32 is a cross-sectional view of the assembled trial humeral head and trial offset adapter taken along line 32-32 in FIG. 31, according to an embodiment of the present disclosure;

FIG. 33 is a perspective view of the assembled trial humeral head and trial offset adapter of FIG. 31, and a hexalobe driver, according to an embodiment of the present disclosure;

FIG. 34 is a perspective view of the humeral sizer disposed over a pin secured in a resected proximal portion of a humerus of a patient, according to an embodiment of the present disclosure;

FIG. 35 is a top perspective view of the humeral sizer of FIG. 34, according to an embodiment of the present disclosure;

FIG. 36 is a bottom perspective view of the humeral sizer of FIG. 35, according to an embodiment of the present disclosure; and

FIG. 37 is a flowchart of a surgical method, according to an embodiment of the present disclosure.

### DETAILED DESCRIPTION

Generally stated, disclosed herein are offset adapters, implant systems, trial implant systems, and methods for orthopedic surgery for replacing an articulation joint, such as, for example, in repairing a shoulder joint of patient. The offset adapters, implant systems, trial implant systems, and methods allow a surgeon to select the eccentricity of a humeral head relative to a humeral anchor based on a patient’s anatomy. As will be appreciated from the present description, the technique of the present disclosure allows the surgeon to select a specific offset adapter from a plurality of different offset adapters that upon assembly with a humeral head, create different eccentricities for use in a humeral head implant system based on the patient’s specific anatomy. The technique of the present disclosure reduces the number of humeral heads and trial humeral heads required compared to conventional systems, which typically require many different lines of humeral heads and trial humeral heads with different eccentricities, resulting in excessive inventory and increasing the complexity of the procedure of repairing a patient’s shoulder.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in the first figure of each embodiment.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be

changed, varied, modified, reconfigured or otherwise altered for use or association with another side of the body for a same or similar purpose without departing from the spirit and scope of the invention. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the lower extremity, and more specifically, with the bones of the ankle, foot, and leg.

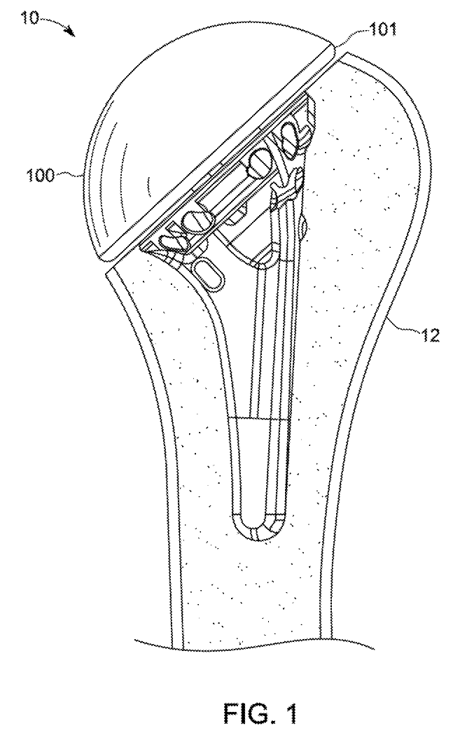

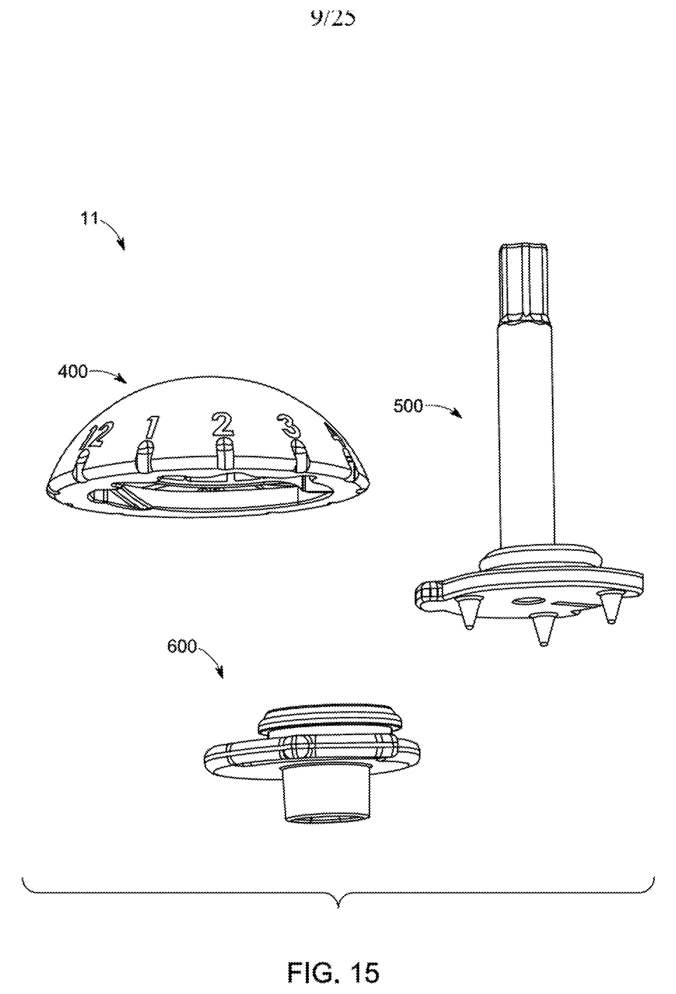

Referring to the drawings, wherein like reference numerals are used to indicate like or analogous components throughout the several views, and with particular reference to FIGS. 1-3, therein illustrated is an exemplary embodiment of a biocompatible humeral head implant system 10 for use in a total shoulder replacement system, according to an embodiment of the present disclosure. FIGS. 15-18 illustrate an exemplary embodiment of a trial humeral head and pin guide for use in selecting a trial humeral head and installing a pin, according to an embodiment of the present disclosure. FIGS. 15, 27, and 28 illustrate an exemplary embodiment of the trial humeral head and a trial offset adapter for use in determining the eccentricity and orientation of a selected trial humeral head and an offset adapter, according to an embodiment of the present disclosure. As will be appreciated from the description below, a technique of the present disclosure may allow a surgeon to implant a humeral head with the selection of the eccentricity of the humeral head that matches the resected humerus relative to the humeral anchor component. The technique of the present disclosure may allow a surgeon to trial with a single component, which assists the surgeon with selecting a certain eccentricity. Then, the definitive implant can be built to the previously determined eccentricity. For example, a single humeral head and a plurality of offset adapters having, e.g., dual tapers, allows various choices of eccentricity achievable when assembled in a humeral head implant system. Such a technique may allow a surgeon to reconstruct a shoulder more quickly and closely replicating the natural biomechanics of a normal shoulder compared to a conventional total shoulder replacement.

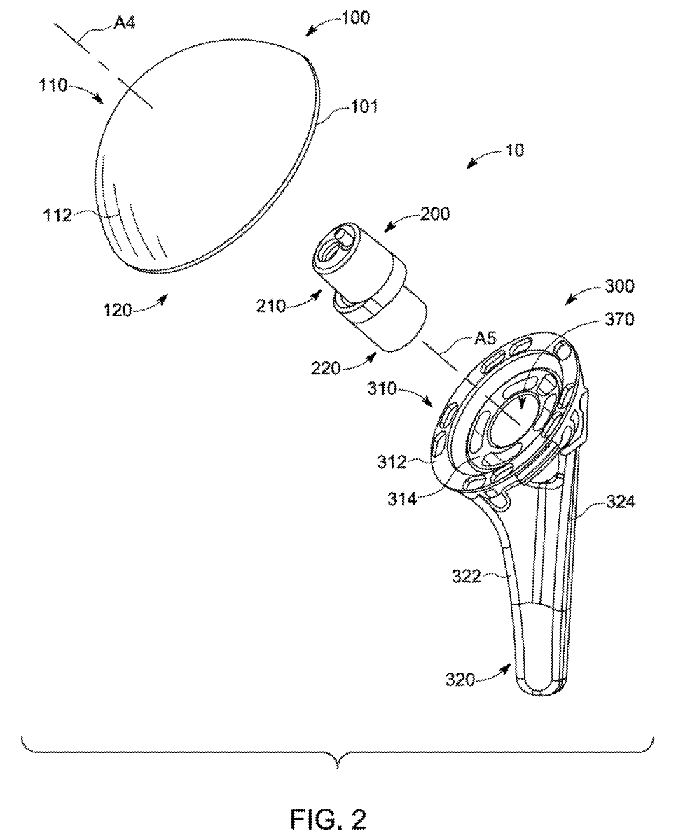

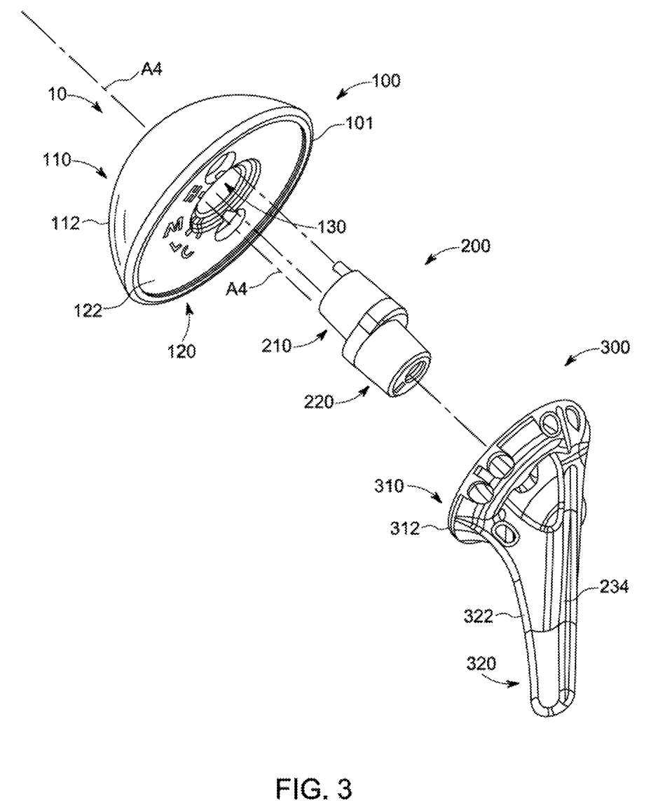

With reference again to FIG. 1, in this exemplary embodiment, the humeral head implant system 10 may be operably attached to a resected proximal portion of a humerus 12 of a patient. As shown in FIGS. 2 and 3, the humeral head implant system 10 may include a humeral head 100, an offset adapter 200, and a humeral anchor 300.

As described in greater detail below, in this illustrated embodiment, the humeral head 100 may have an offset cavity 130 (FIG. 3), the offset adapter 200 may have a first portion 210 and an offset second portion 220, and the humeral anchor 300 may have a centrally disposed cavity 370 (FIG. 2). The combination of the configuration of the humeral head 100, the offset adapter 200, and the humeral anchor 300 is operable to aid in eccentrically positioning the humeral head 100 relative to the resected proximal portion 12 (FIG. 1) of the humerus of the patient. When the humeral anchor 300 is installed in the resected proximal humerus 12 (FIG. 1) of a patient, the humeral head 100 and the offset adapter 200, selected from a plurality of offset adapters having different offsets as described below, allows a surgeon to use a single configured humeral head to be disposed over a range of positions to optimize the position of a peripheral edge 101 (FIG. 1) of the humeral head 100 (FIG. 1) relative to the resected proximal portion of the humerus 12 (FIG. 1) of the patient and balance the surrounding soft tissue.

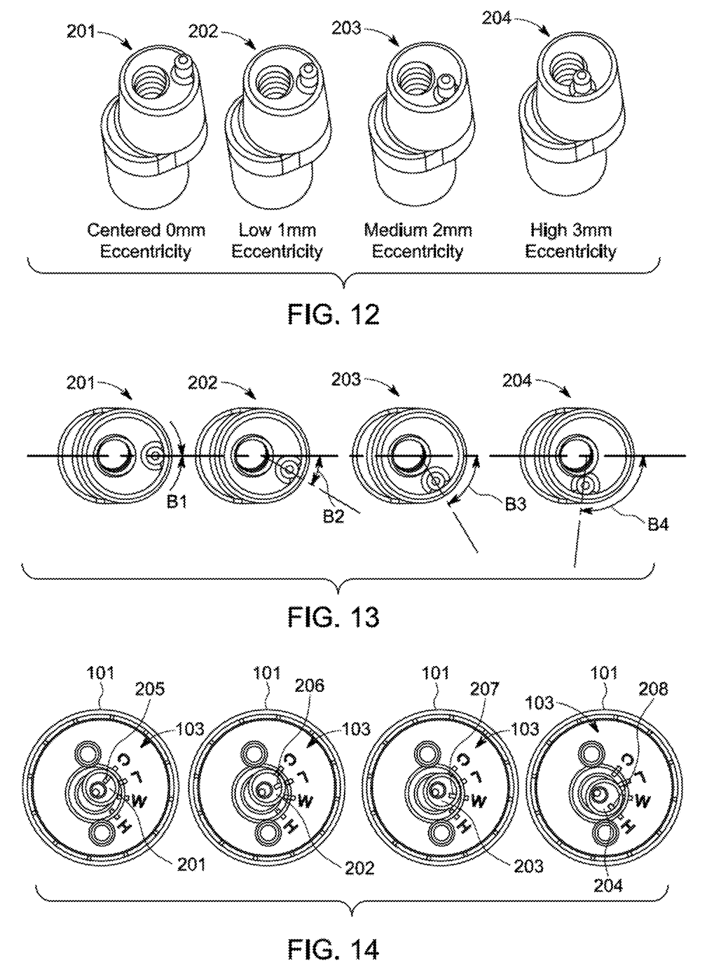

As described in greater detail below, a plurality of different offset adapters, such as those shown in FIGS. 12 and 13, may be provided, and upon a trialing procedure also described in greater detail below, a surgeon may determine and select one of a plurality of humeral heads and one of a plurality of offset adapters so that the eccentricity of the humeral head relative to the humeral anchor and resected proximal humerus may be optimally positioned on a resected proximal portion of a humerus of a patient. As will be appreciated from the present description, a reduced number of differently sized and configured humeral heads and a plurality of differently configured offset adapters may be required for suitably positioning a humeral head on a plurality of different patients compared to the number of conventional humeral heads having specific eccentricities required that are used with a monoblock offset taper.

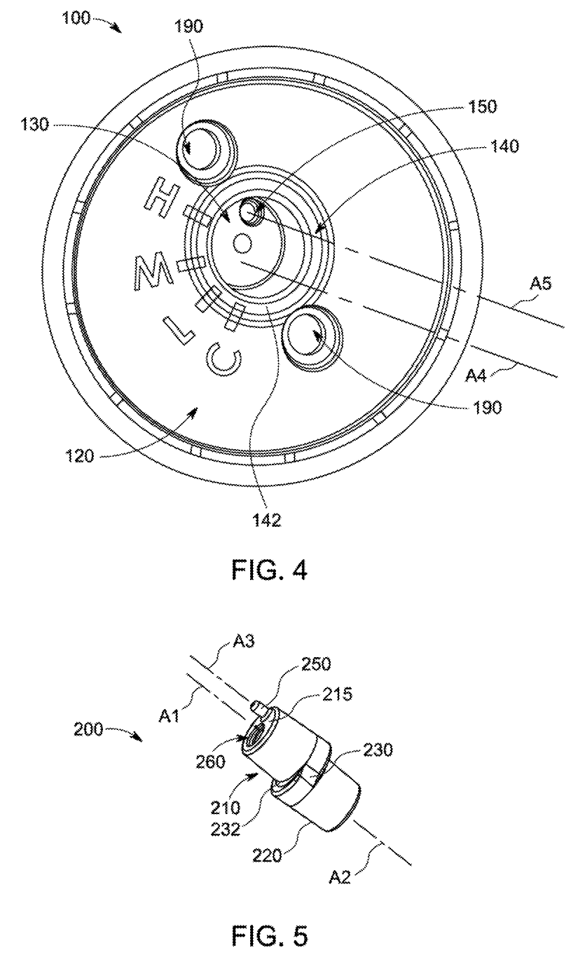

In addition, as shown in FIGS. 4 and 5, the humeral head 100 (FIG. 4) and the offset adapter 200 (FIG. 5) may be fixedly coupled to each other. For example, the offset adapter 200 (FIG. 5) may include a projection 250 (FIG. 5) which is receivable in a recess 150 (FIG. 4) in the humeral head 100 (FIG. 4) to aid in fixing the offset adapter 200 (FIG. 5) from rotation relative to the humeral head 100 (FIG. 4). As described below, the assembled humeral head and the offset adapter 200 may be aligned in a designed rotated position relative to the humeral anchor 300 (FIG. 2) and then secured to the humeral anchor 300.

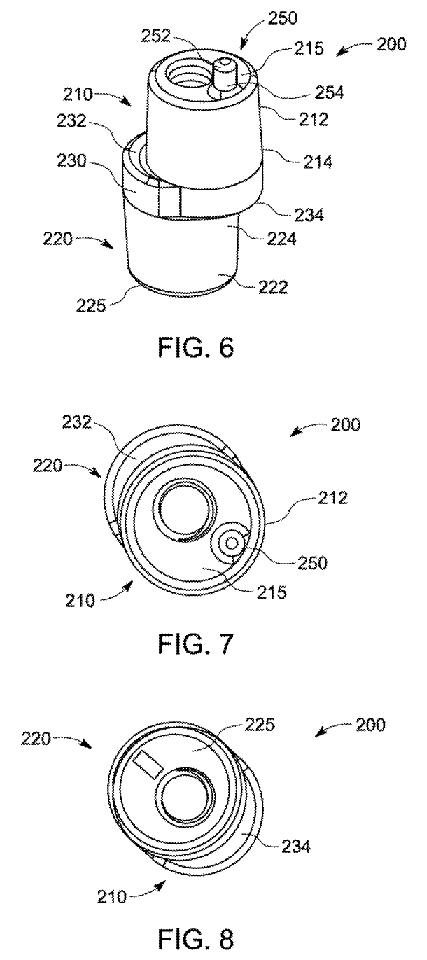

With reference to FIGS. 6-8, the offset adapter 200 may include a first portion 210 and an offset second portion 220 positioned opposite the first portion 210. The first portion 210 may include an upper distal end 212 and a lower proximal end 214. The upper distal end 212 may include an upper surface 215. The second portion 220 may include a lower distal end 222 and an upper proximal end 224. The lower distal end 222 may include a lower surface 225. The proximal end 214 of the offset adapter 200 may be operably jointed to the proximal end 224 of the offset adapter 200 along an oblong midsection 230 defining a first stop 232 and an opposite stop 234.

As shown in FIG. 7, the projection 250 extends away from the distal surface 215. The projection 250 may have a distal end 252 (FIG. 6) and a proximal end 254 (FIG. 6) attached to the upper surface 215 of the upper distal end 212 of the first portion 210 of the offset adapter 200.

With reference again to FIG. 5, the first portion 210 may define a first central axis A1 and the second portion 220 may define a second central axis A2. The first central axis A1 may be offset from or eccentrically disposed relative to the second central axis A2. The projection 250 extending away from the distal surface 215 defines a central axis A3. The central axis A3 may be offset from or eccentrically disposed relative to the first central axis A1 of the first portion 210 of offset adapter 200 and may be offset from or eccentrically disposed relative to the second central axis A2 of the second portion 220 of the offset adapter 200.

In some embodiments, the first portion 210 may be a tapered cylindrical portion or have a frustoconical configuration, and the second portion 220 of the offset adapter 200 may be a tapered cylindrical portion or have a frustoconical configuration. The configuration, size, shape of the first portion 210 of the offset adapter 200 and the second portion 220 of the offset adapter 200 may be the same. In other embodiments, the first portion 210 and the second portion 220 of the offset adapter 200 may be configured differently. The offset adapter 200 may also include a through hole or threaded opening 260 extending through the offset adapter 200 from the first portion 210 to the second portion 220.

With reference again to FIGS. 2 and 3, the humeral head 100 may include a first portion 110 and a second portion 120. The first portion 110 of the humeral head 100 may have an articular surface 112. The second portion 120 of the humeral head 100 may be a coupling portion having a second or bottom surface 122 (FIG. 3) positioned opposite to the articular surface 112.

The articular surface 112 may be a convex surface such as a surface of a hemisphere of a spherical cap or alternatively, a non-spherical surface. The first portion 110 and the second portion 120 of the humeral head 100 may define a central axis A4 extending from the first portion 110 to the second portion 120 of the humeral head 100. The second portion or coupling portion 120 may include the recessed opening 130 (FIG. 3) such as a tapered cavity therein extending into the second or bottom surface 122 towards the articular surface 112.

As shown in FIG. 4, the second or coupling portion 120 may include, for example, a recessed region 140 that forms an outer circumferentially-extending recessed lip 142 surrounding the recessed cavity 130. The recessed region 140 may be, for example, sized and shaped to receive and engage the stop 232 (FIG. 5) of the oblong midsection 230 (FIG. 5) of the offset adapter 200 (FIG. 5). The two circular threaded cavities 190 are provided and employed in the fabrication of the humeral head 100.

The cavity 130 may include a recessed opening 150 defining an axis A5 offset and eccentrically disposed relative to the central axis A4 of the humeral head 100. Recessed opening 150 is operable for receiving the projection 250 (FIG. 5) of the offset adapter 200 (FIG. 5) when the first tapered cylindrical portion 210 (FIG. 5) of the offset adapter 200 (FIG. 5) is positioned in and received into the tapered cavity 130 of the humeral head 100 with the projection 250 (FIG. 5) being located in the recess 150 in the tapered cavity 130 of the humeral head 100.

Referring again to FIGS. 2 and 3, in this illustrated embodiment, the humeral anchor 300 may be a humeral stem anchor. The humeral anchor 300 may include a first proximal portion or base 310 and a second portion or stem 320. As shown in FIG. 2, the base 310 may include an outer ring 312 and a recessed portion 314 having the cavity 350 disposed herein. The base 310 may be configured as a ring or surface area to assist with fixation, for example, the base 310 may contact cancellous bone to provide better post-operative support for the humeral head implant system 10 (FIG. 1). The stem 320 may also include a fin, leg, or elongated protrusion 322 extending away from the at least one interior surface. The fin 322 may be positioned, for example, at a midpoint or midline of the stem 320. The stem may have, for example, a T-shape cross-section. The stem 320 may be, for example, tapered as it extends away from the base 310. In other embodiments, the humeral anchor may be a stemless humeral anchor. Suitable humeral stem anchors and suitable humeral stemless anchors for use in the humeral head implant systems of the present disclosure may include the humeral stem anchors and the humeral stemless anchors described in International PCT Patent Application Publication No. WO 2020/023971 by Hodorek et al., the entire subject matter of which is incorporated in its entirety herein by reference.

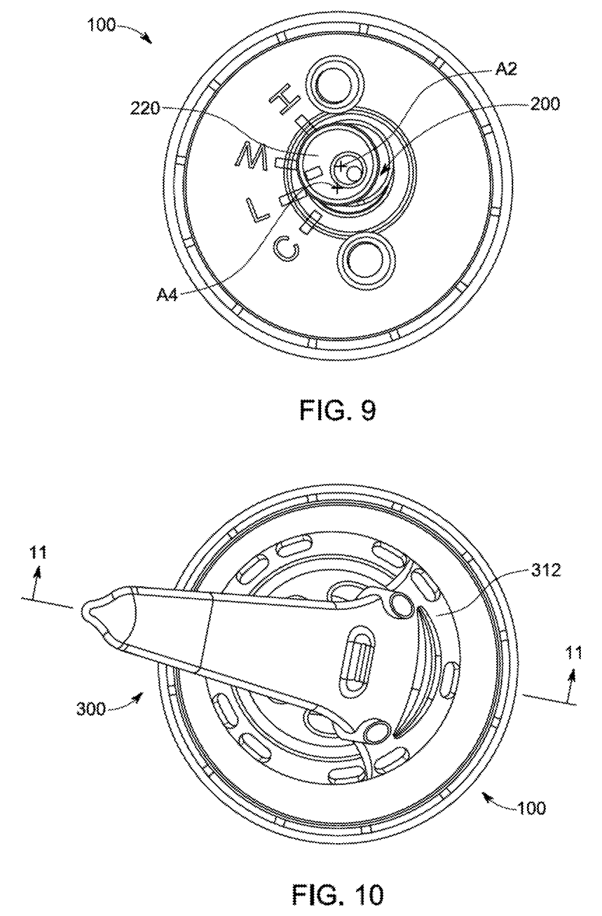

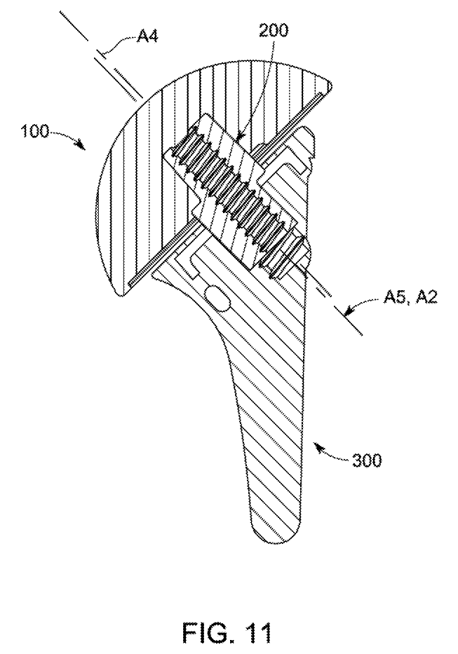

FIG. 9 illustrates the offset adapter 200 secured to the humeral head 100, according to an embodiment of the present disclosure. As illustrated, the position of the projection 250 (FIG. 5) relative to the first axis A1 (FIG. 5) of the first tapered cylindrical portion 210 (FIG. 5) and to the second axis A2 (FIG. 5) of the second tapered cylindrical portion 220 is operable to provide the central axis A4 of the humeral head 100 disposed eccentrically relative to the axis A2 of the second portion 220 of the offset adapter 200. Accordingly, as shown in FIG. 10, the humeral head 100 may be disposed eccentrically relative to the base 312 of the humeral anchor 300. For example, as shown in FIG. 11, the central axis A4 of the humeral head 100 may be offset from a central axis A5 (also shown in FIG, 2) of the humeral anchor 300, which when the offset adapter 200 is secured to the humeral anchor 300, axis A2 of the offset adapter 200 is aligned with central axis A5 of the humeral anchor 300. As described in greater detail below, a plurality of offset adapters 200 may be provided in a kit in which the projection is positioned differently to allow for greater, lesser, or no eccentricity relative to the central axis of the humeral head 100, and to the central axis of the humeral anchor 300.

As shown in FIG. 12, a plurality of offset adapters 201, 202, 203, and 204 may be provided for use by a surgeon in selecting a desired eccentricity of the humeral head 100 relative to the humeral anchor 300, according to embodiments of the present disclosure. For example, the offset adapter 201 may be operable for centering, no eccentricity, or aligning the central axis of the humeral head 100 to the central axis of the cavity of the humeral anchor 300. The offset adapter 202 may be operable for low eccentricity or 1 millimeter (mm) eccentricity. The offset adapter 203 may be for operable for medium eccentricity or 2 mm eccentricity. The offset adapter 204 may be for operable for high eccentricity or 3 mm eccentricity. As shown in FIG. 13, the offset adapters 201, 202, 203, and 204 may be essentially the same with the exception that the projections 250 being disposed at different angular positions, e.g., B1, B2, B3, and B4, relative to the offset upper and lower portions of the offset adapters 201, 202, 203, and 204, respectively. FIG. 14 illustrates the offset adapters 201, 202, 203, and 204 secured to a humeral head 101. A marking 205, 206, 207, and 208 may be disposed on the distal end (or side) of offset adapters 201, 202, 203, and 204, respectively, and alignable with indicia “C”, “L”, “M”, and “H” on humeral head 101.

As described further below, before final installation to secure the humeral head implant system 10 to the resected proximal humerus 12, the eccentrically disposed humeral head 100 may be rotated or aligned relative to the resected proximal humerus 12, e.g., the outer peripheral portion of the humeral head may be marked and aligned with the most lateral position of the resected proximal portion of the humerus.

With reference to FIG. 15, in this exemplary embodiment, a trial humeral head system 11 may include a trial humeral head 400, a pin guide 500, and a trial offset adapter 600 according to an embodiment of the present disclosure. As described in greater detail below, the trial humeral head 400 and the pin guide 500 are used for installing the humeral anchor 300, and the trial humeral head 400 and the trial offset adapter 600 are used for trialing the trial humeral head 400 and selecting the appropriate offset adapter 200.

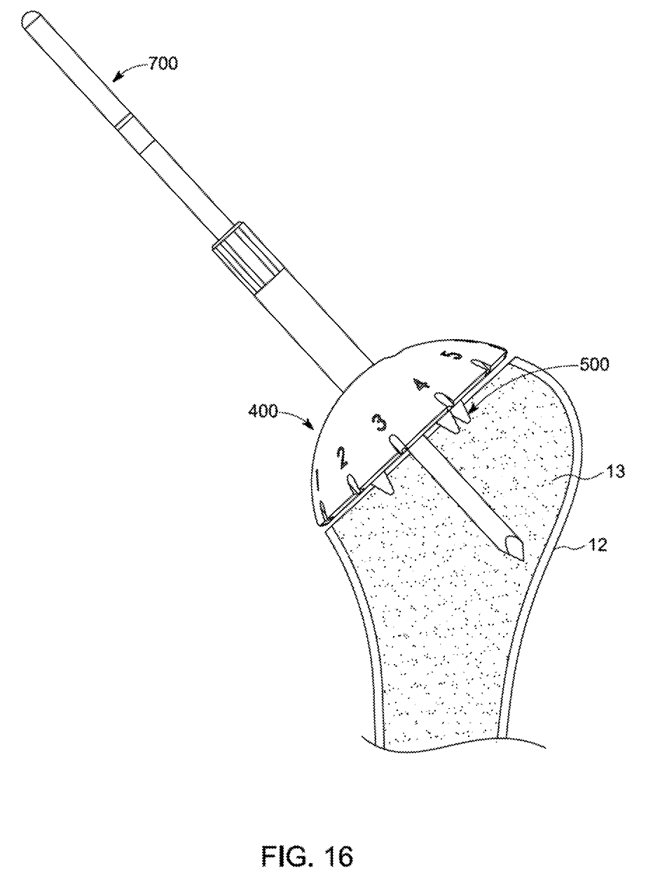

As shown in FIG. 16, the trial humeral head 400 and the pin guide 500 are assembled and positioned on a resected proximal portion of a humerus 12 of the patient to guide a pin 700 into the cancellous bone 13 of the resected humerus 12.

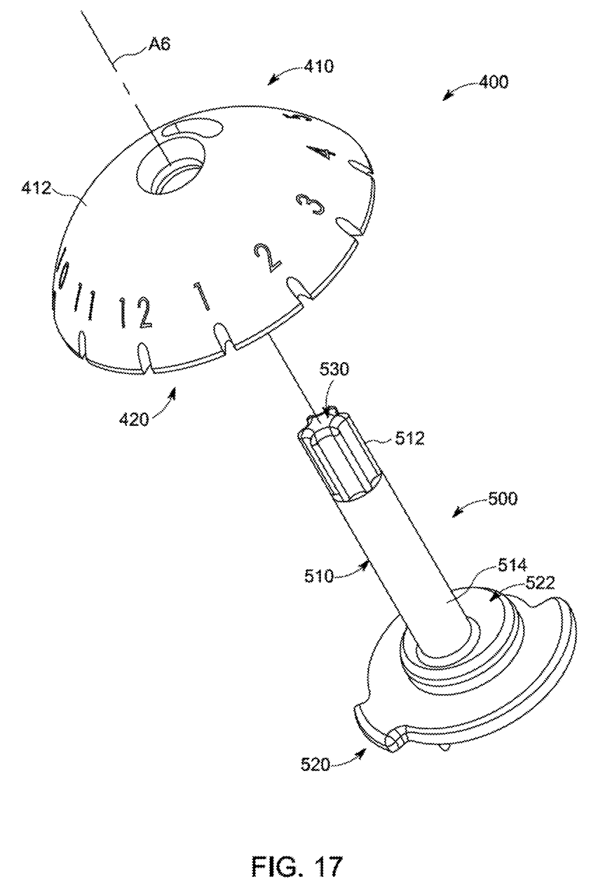

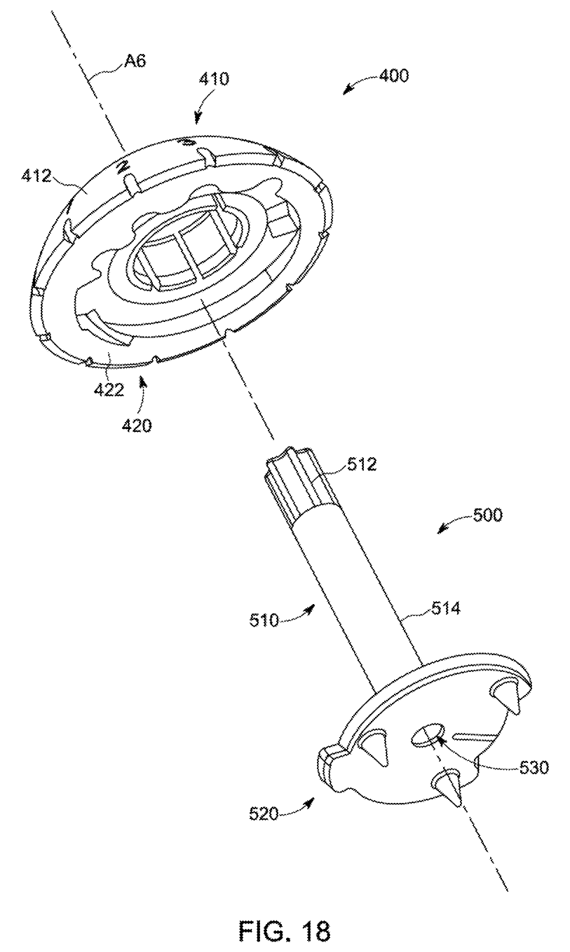

With reference to FIGS. 17 and 18, the trial humeral head 400 may include a first portion 410 and a second portion 420. The first portion 410 of the trial humeral head 400 may have an articular surface 412. The second portion 420 of the trial humeral head 400 may be a coupling portion having a second or bottom surface 422 (FIG. 18) positioned opposite to the articular surface 412.

The articular surface 412 may be a convex surface such as a surface of a hemisphere of a spherical cap, or alternatively, a non-spherical cap. The first portion 410 and the second portion 420 of the trial humeral head 400 may define a central axis A6 extending from the first portion 410 to the second portion 420 of the trial humeral head 400. The second portion or coupling portion 420 may be configured for receiving the pin guide 500 as well as the trial offset adapter 600 (FIG. 15).

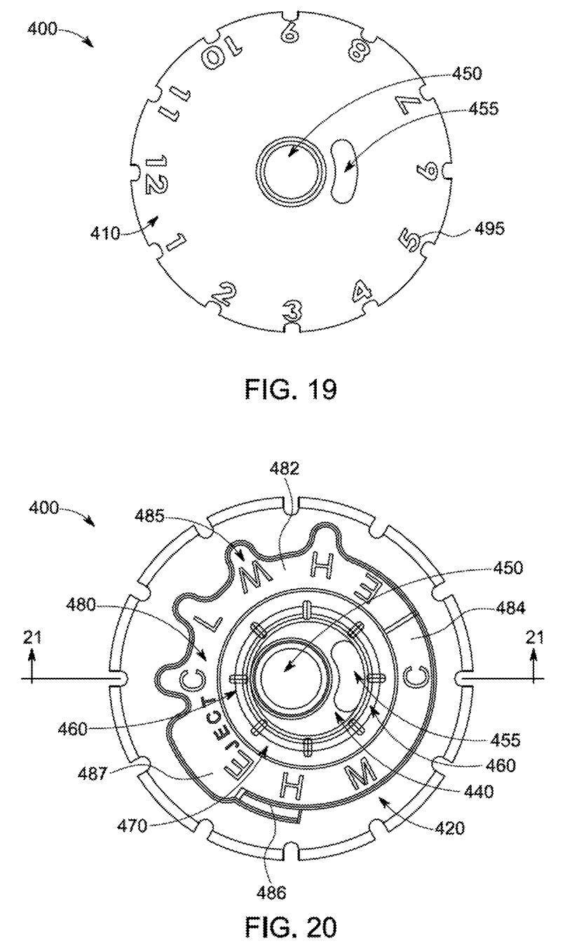

As shown in FIGS. 19 and 20, the trial humeral head 400 may include a central opening 450 extending from the first portion 410 (FIG. 19) to the second portion 420 (FIG. 20). An oblong opening 455 may extend from the first portion 410 (FIG. 19) to the second portion 420 (FIG. 20) and be disposed adjacent to the central opening 450.

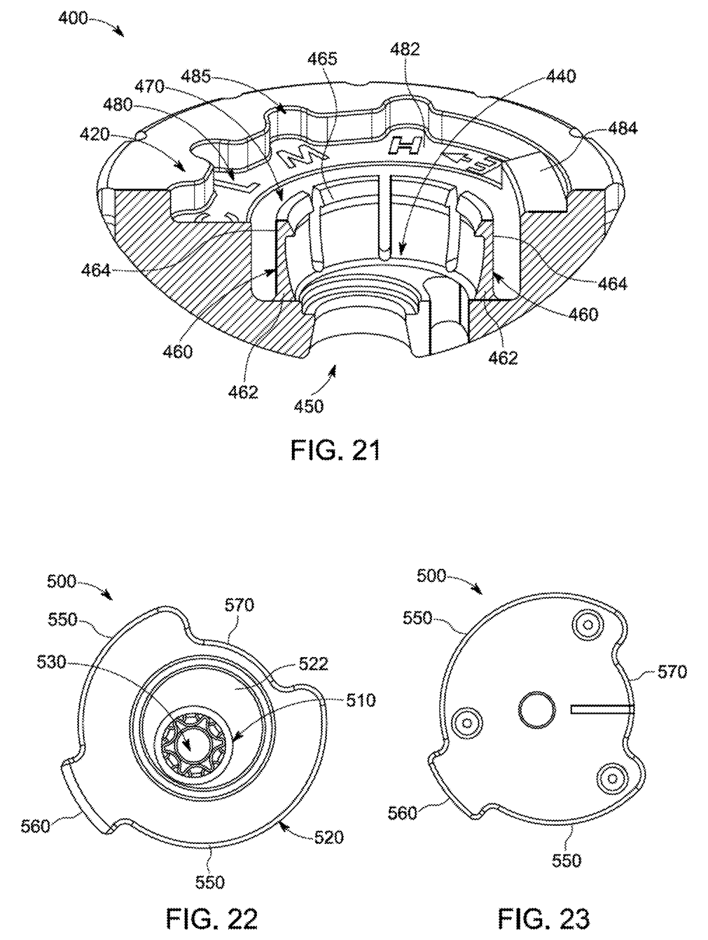

As shown in FIGS. 20 and 21, the second or coupling portion 420 may include, for example, an eccentric cavity 440 defined by a plurality of resilient fingers 460 having a proximal portion 462 (FIG. 21) and a distal portion 464 (FIG. 21). The distal portions include inwardly extending projections 465 (FIG. 21). An annular channel 470 is disposed around the plurality of resilient fingers 460. The annular channel 470 extends from the proximal portion of the fingers to the distal portion of the fingers. The annular channel 470 allows the plurality of fingers 460 to flex outwardly to releasably connect to the pin guide 500 (FIG. 15) in a snap-fit manner and to releasably connect to the trial offset adapter 600 (FIG. 15) in a snap-fit manner.

The second or coupling portion 420 may include an outer circumferentially-extending recessed ledge 480 surrounding the annular channel 470 and the recessed cavity 440. The recessed ledge 480 may be, for example, sized and shaped to receive a peripheral portion of the guide pin 500 and the trial offset adapter 600.

The recessed ledge 480 may include a planar surface portion 482 and a first angled or ramp portion 484 (best shown in FIG. 21) and a second angled or ramp portion 486. The recessed ledge 480 may include a plurality of radially outwardly-extending semi-circular portions 485 for use in positioning and orienting the trial offset adapter 600 (FIG. 15) as described below. The recessed ledge 480 may include indicia disposed adjacent to the plurality of radially outwardly-extending semi-circular portions 485. The indicia may be a letter “C” to identify centered or zero eccentricity, a letter “L” to identify low eccentricity, a letter “M” to identify medium eccentricity, and a letter “H” to identify high eccentricity. The recessed ledge 480 may include a radially outwardly-extending rectangular portion 487 (FIG. 20) for use in positioning and orienting the pin guide 500 (FIG. 15) in a single fixed position and orientation relative to the trial humeral head 400 as described below.

With reference again to FIGS. 17 and 18, the pin guide 500 may include an elongated member 510 and an outwardly-extending member 520. The elongated member 510 includes a first or upper end 512, and a second or lower end 514. The lower end 514 of the elongated member 510 is secured to the outwardly-extending member 520. A passageway 530 extends through the elongated member 510 from the first end 512 to the outwardly-extending member 520.

As shown in FIGS. 17 and 22, the outwardly-extending member 520 may have an upper inner disc 522. The upper inner disc 522 is disposed eccentrically compared to the elongated member 510 and the passageway 530. With reference to FIGS. 22 and 23, the outwardly-extending member 520 may have a generally circular outer periphery 550 with an outwardly-extending tab 560 and an inwardly-extending cutout 570.

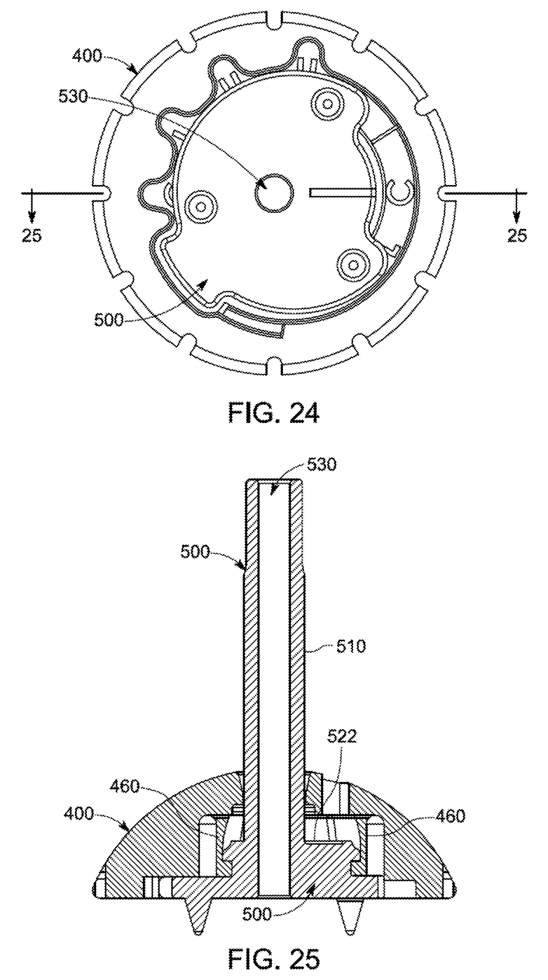

FIGS. 24 and 25 illustrate the assembled trial humeral head 400 and the trial offset adapter 500. The upper inner disc 522 (FIG. 25) is disposed eccentrically between fingers 460 (FIG. 25) compared to the elongated member 510 (FIG. 25) and the passageway 530. The outwardly-extending member is releasably securable in the eccentric cavity of the trial humeral head 400 with the elongated member 510 extending through the aperture of the trial humeral head 400 and with the passageway 530 of the pin guide 500 concentric with the central axis of the trial humeral head 400.

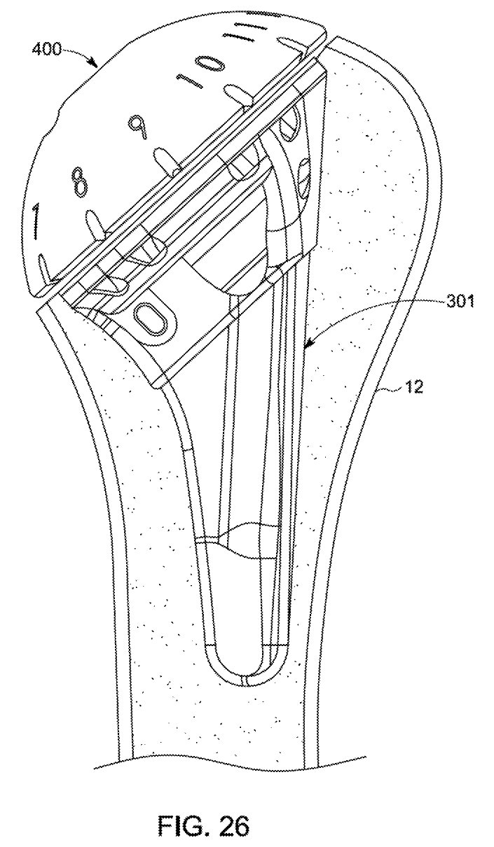

As shown in FIG. 26, in this exemplary embodiment, the trial humeral head 400 and the trial offset adapter 600 (FIG. 15) may be operably connectable to a humeral anchor 301 and positioned on a resected proximal portion of the humerus 12 of the patient, according to an embodiment of the present disclosure. The trial humeral head 400 and the trial offset adapter 600 (FIG. 15) are used for trialing the humeral head 100 (FIG. 1) and selecting the appropriate offset adapter 200.

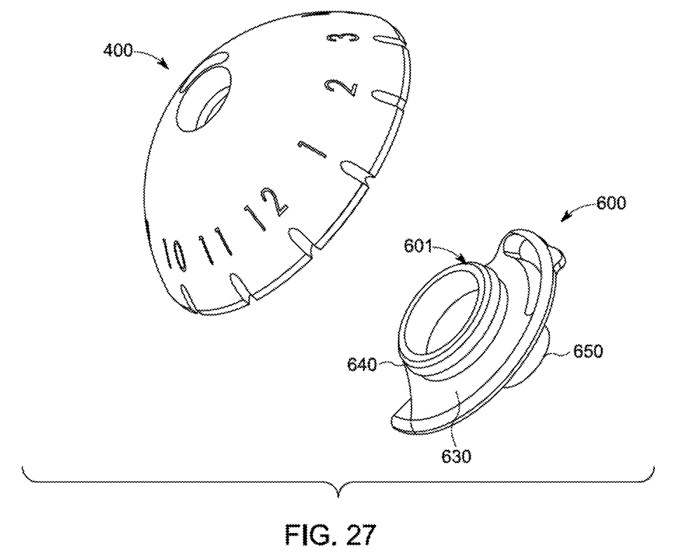

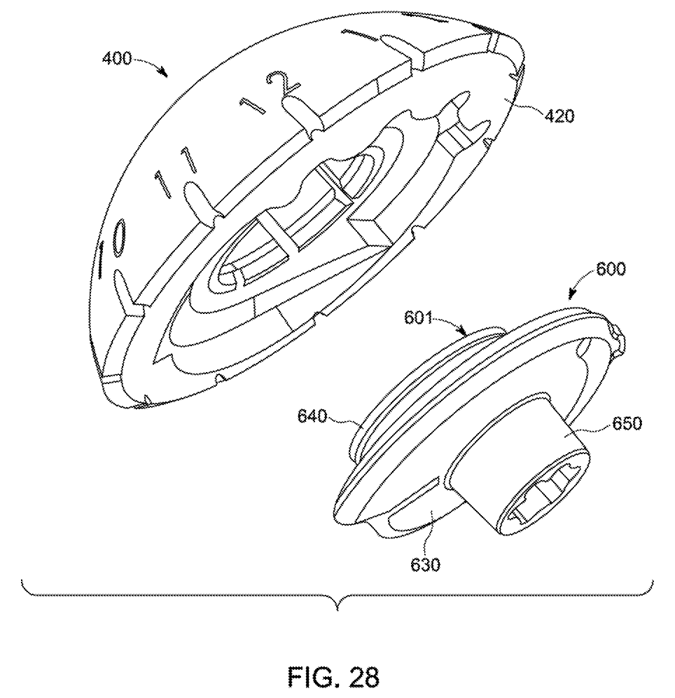

With reference to FIGS. 27 and 28, the second portion or coupling portion 420 (FIG. 28) of the trial humeral head 400 may be configured for receiving the trial offset adapter 600.

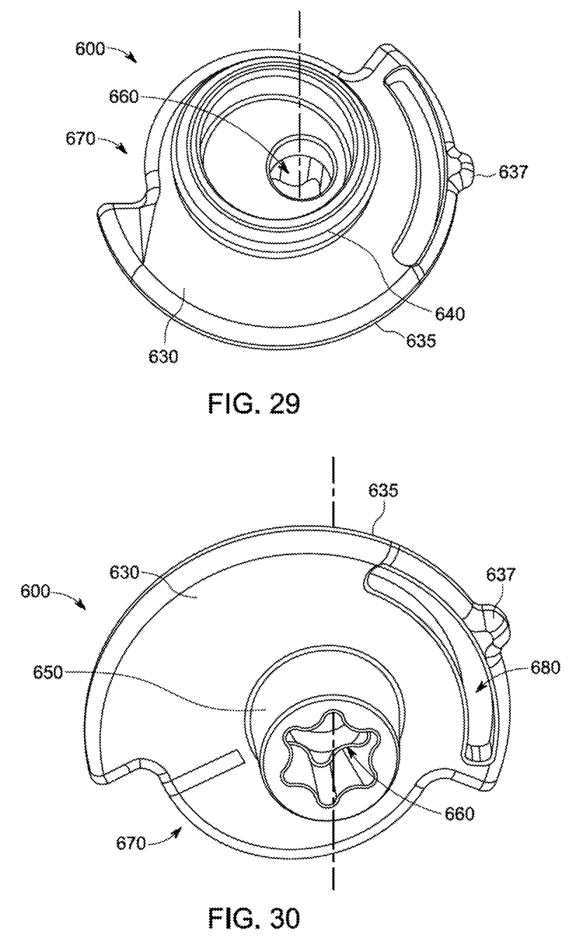

The trial offset adapter 600 may include a body 601 having a central outwardly-extending generally disc-shaped member 630, an upper inner ring 640, and a lower tapered portion 650. As shown in FIGS. 29 and 30, a passageway 660 extends through the trial offset adapter 600 from the upper inner ring 640 to the lower tapered portion 650 (FIG. 30).

The outwardly-extending member 630 may have a generally circular outer periphery 635 with an outwardly-extending detent 637 and an inwardly-extending cutout 670. As shown in FIG. 29, the upper inner ring 640 is disposed eccentrically compared to the central outwardly-extending generally disc-shaped member 630 and the passageway 660. As shown in FIG. 30, the lower tapered portion 650 is disposed eccentrically compared to the central outwardly-extending generally disc-shaped member 630 and the passageway 660. A curved channel 680 may extend through the central outwardly-extending generally disc-shaped member 630 to allow the detent 637 to be movable inwardly.

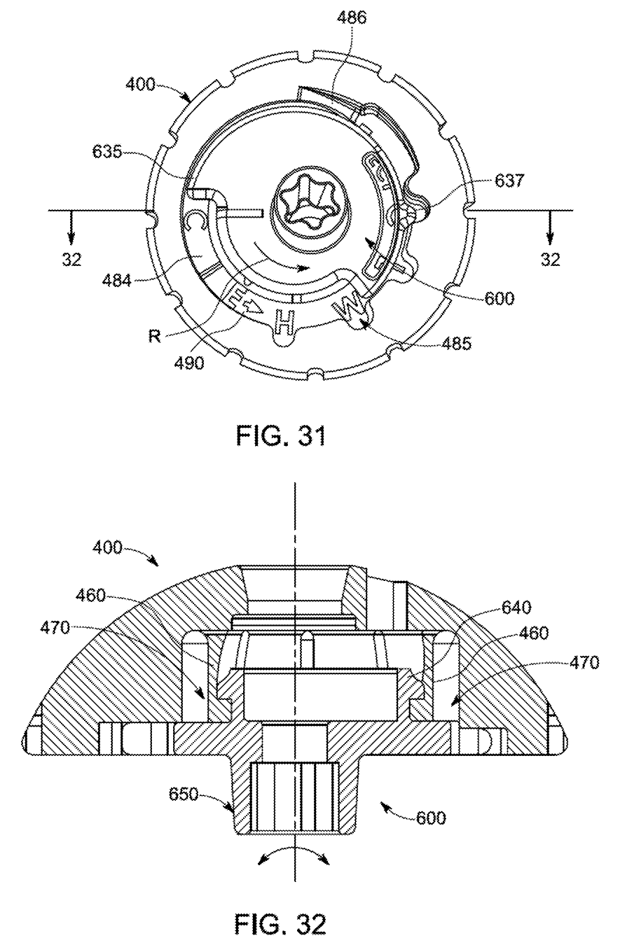

FIGS. 31 and 32 illustrate the assembled trial humeral head 400 and the trial offset adapter 600. The upper inner ring 640 (FIG. 32) is disposed between the plurality of fingers 460 (FIG. 32). The outwardly-extending generally disc-shaped member 630 is releasably securable against the outer circumferentially-extending recessed ledge 480 (FIG. 21) of the trial humeral head 400 with the lower tapered portion 650 extending downwardly. The annular channel 470 (FIG. 32) allows the plurality of fingers 460 to flex outwardly to releasably connect to the trial offset adapter 600 (FIG. 15) in a snap-fit manner and to releasably connect to the trial offset adapter 600 (FIG. 15) in a snap-fit manner. As shown in FIG. 31, the trial humeral head 400 may include an indicia 490 such as the letter “E” and an arrow indicating the direction of rotation of the trial offset adapter 600 relative to the trial humeral head 400 so that the trial offset adapter 600 may be disconnected from the trial humeral head 400. As the trial offset adapter 600 is sufficiently rotated in the direction R, the generally circular outer periphery 635 of the outwardly-extending member 630 engage ramp 484, and the detent 637 may engage ramp486 to cause and force the upper disc 640 of the trial offset adapter 600 to pop out of the resilient fingers 460 (FIG. 32).

The trial humeral head 400 is used to determine the curvature and diameter required for the resected proximal portion of the humerus 12 of the patient. The trial offset adapter 600 is rotatable or dialable by engaging the detent 637 with one of the four radially outwardly-extending semi-circular portions 485 (FIG. 31) having indicia such as the letter “C” to identify centered or zero eccentricity, the letter “L” to identify low eccentricity, the letter “M” to identify medium eccentricity, and the letter “H” to identify high eccentricity.

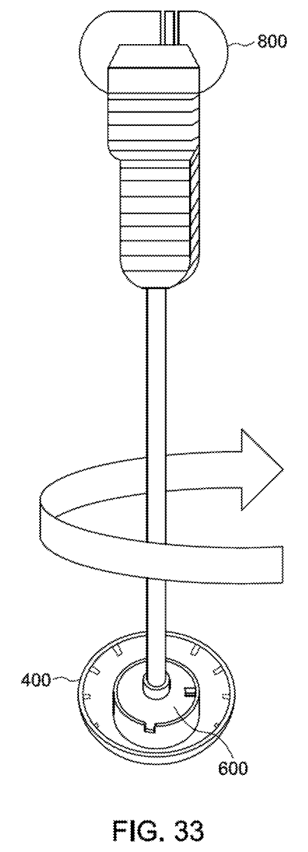

As shown in FIG. 33, the trial offset adapter 600 may be rotated relative to the trial humeral head 400, using a hexalobe tool 800, so that the detent 637 (FIG. 31) clicks into one of the four radially outwardly-extending semi-circular portions 485 and so that the lower tapered portion 650 (FIG. 32) is disposed at different eccentricities relative to the central axis of the trial humeral head 400. The surgeon can select a degree of eccentricity and assemble it to the humeral anchor 300 (FIG. 1), and if necessary, remove it and adjust the eccentricity of the trial humeral head 400 relative to the trial offset adapter 600. When the desired eccentricity is achieved, the offset and the orientation of the trial humeral head 400 may be recorded. For example, the offset may be a center or no eccentricity, or a low, a medium, or a high eccentricity. The orientation of the trial humeral head 400 may employ indicia 495 (FIG. 19) disposed around the periphery of the outer surface of the trial humeral head 400 (FIG. 19) that is aligned with the most lateral position of the resected proximal

portion of the humerus 12 of the patient. The correct humeral head size may be selected based on the diameter and thickness previously used in trialing.

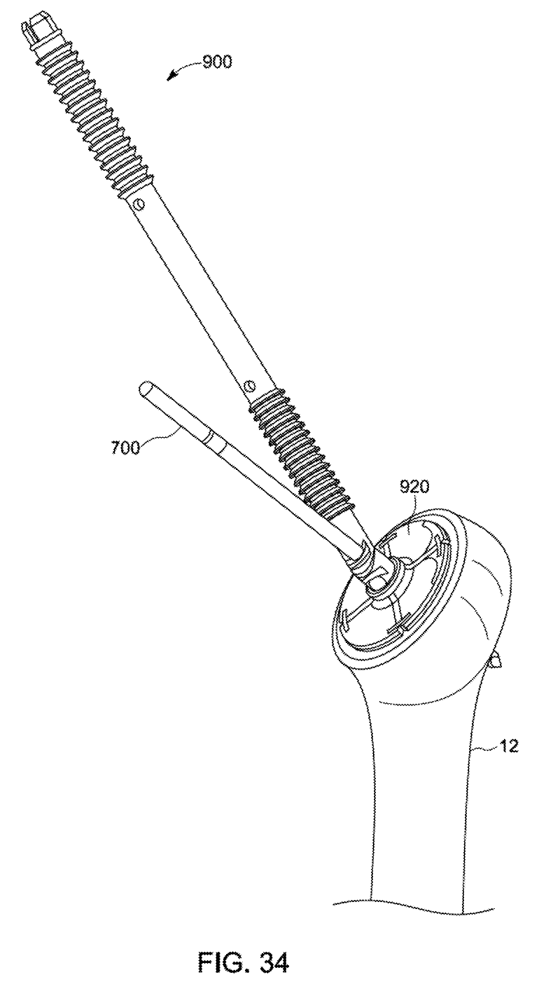

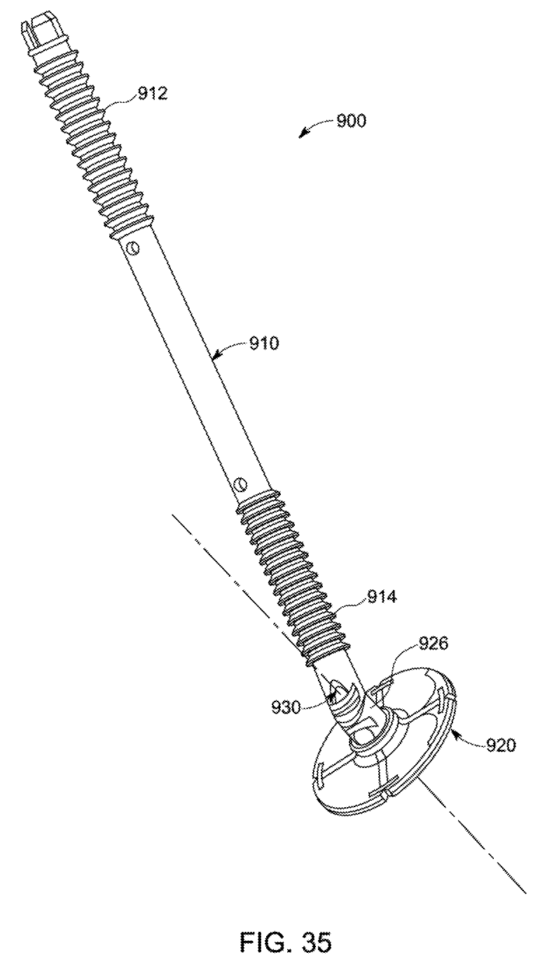

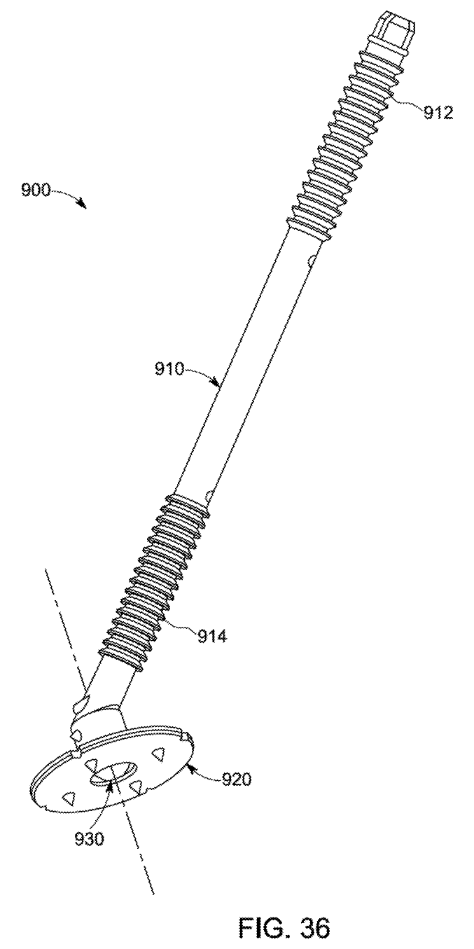

FIG. 34 illustrates a humeral head sizer 900 disposed on a resected proximal portion of a humerus 12 of a patient, according to an embodiment of the present disclosure. As shown in FIGS. 35 and 36, the humeral head sizer 900 may include a handle 910 and a base plate 920. The distal end 914 of the handle 910 and the base plate 920 may include a passageway 930 extending therethrough. The passageway 930 is extendable over pin 700 (FIG. 34). A surgeon is able to select the largest sized implant based on the size of the base plate 920 appropriate for the patient’s anatomy being careful not to place the periphery of the base plate 920 on the resected cortical bone. The outside of the humeral head sizer 900 is desirably selected to cover most of the cancellous bone, without involving the cortex. It may be desirable to allow for a 5 mm ring of bone from the outside of the cortex to the humeral head sizer peripheral edge of the base plate 920 for proper sizing of a humeral anchor 300 such as for example, a humeral stemmed anchor or a humeral stemless anchor.

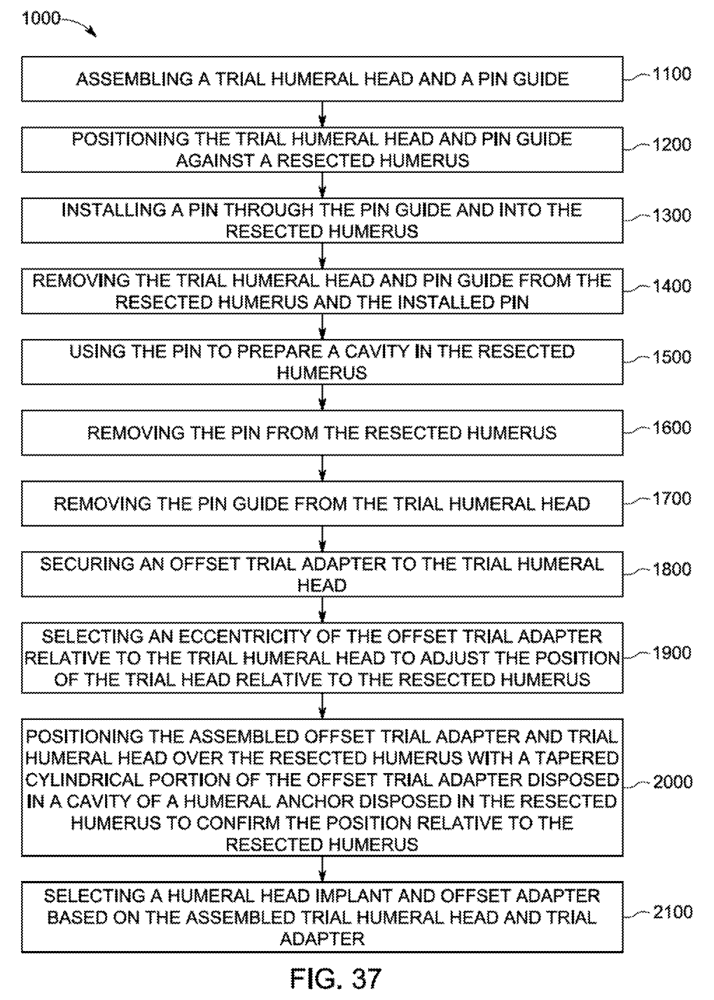

FIG. 37 illustrates a surgical method 1000 for sizing a humeral head relative to a humeral anchor, in which the humeral anchor is disposable in a proximal portion of a humerus of a patient. The method 1000 may include, for example, at 1100 assembling a trial humeral head and a pin guide, at 1200 positioning the trial humeral head and pin guide against a resected humerus, at 1300 installing a pin through the pin guide and into the resected humerus, at 1400 removing the trial humeral head and pin guide from the resected humerus and the installed pin, at 1500 using the pin to prepare a cavity in the resected humerus, at 1600 removing the pin from the resected humerus, at 1700 removing the pin guide from the trial humeral head, at 1800 securing an trial offset adapter to the trial humeral head, at 1900 selecting the eccentricity of the trial offset adapter relative to the trial humeral head to adjust the position of the trial humeral head relative to the resected humerus; at 2000 positioning the assembled trial offset adapter and the trial humeral head over the resected humerus with a tapered cylindrical portion of the trial offset adapter disposed in a cavity of a humeral anchor disposed in the resected humerus to confirm the position relative to the resected humerus, and at 2100 selecting a humeral head and offset adapter based on the assembled trial humeral head and trial offset adapter.

From the present description it will be appreciated that the technique of the present disclosure utilizing a humeral head and offset adapter that employs a peg-in-hole system, provides a cost-effective and intuitive humeral head implant system. The present technique provides quick and inexpensive humeral head implant systems that allow for addressing and determining the eccentricity of the humeral head relative to the resected proximal portion of the humerus of a patient.

This present technique allows a surgeon to select the eccentricity in a manner that is not confusing, prevents the taper from being inserted upside down, and is cost efficient. For example, the present technique allows a surgeon to adjust the eccentricity without the ability to be malpositioned.

Conventional humeral heads have predetermined diameters and thickness to address and replicate normal human anatomy. However, having each of the humeral heads in several different eccentricities requires excessive inventory. The present technique provides for a reduced number of predetermined humeral heads along with offset adapters to allow a surgeon to select and customize the eccentricity of the humeral head to replicate each patient’s presented anatomy.

The same trial humeral head may be used for both steps, e.g., with a pin guide to locate a pin, and a trial offset adapter to determine eccentricity, leveraging the same trial humeral heads and providing several options for the surgeon while minimizing the amount of instruments required in performing a total shoulder replacement.

As may be recognized by those of ordinary skill in the art based on the teachings herein, numerous changes and modifications may be made to the above-described and other embodiments of the present disclosure without departing from the scope of the disclosure. The implants, screws, and other components of the devices and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices and systems may include more or fewer components or features than the embodiments as described and illustrated herein. Accordingly, this detailed description of the currently-preferred embodiments is to be taken as illustrative, as opposed to limiting the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

1. A humeral head implant system comprising:
a humeral head comprising a first portion and a second portion, said first portion having an articular surface, said first portion and said second portion defining a central axis extending from said first portion to said second portion, said second portion having a surface with a tapered cavity therein, said cavity having a recess therein, said cavity defining an axis offset and eccentrically disposed relative to said central axis of said humeral head;
an offset adapter comprising a first tapered cylindrical portion having a first axis, a second tapered cylindrical portion having a second axis, and said first axis being eccentrically disposed relative to said second axis of said offset adapter, a distal end of said first tapered cylindrical portion comprising a protrusion offset from said first axis of said first tapered cylindrical portion of said offset adapter and from said second axis of said second tapered cylindrical portion;
a humeral anchor having a tapered cavity defining a central axis;
said first tapered cylindrical portion of said offset adapter being receivable in said tapered cavity of said humeral head with said protrusion being receivable in said recess in said tapered cavity of said humeral head; and
said second portion of said offset adapter being receivable in said cavity of said humeral anchor.

2. The humeral head implant system of claim 1, wherein an angular position of said protrusion relative to said first axis of said first tapered cylindrical portion and to said second axis of said second tapered cylindrical portion is operable to provide said central axis of said humeral head being eccentrically disposed relative to said central axis of said humeral anchor.

3. The humeral head implant system of claim 1, wherein said offset adapter comprises a plurality of said offset adapters, each of said plurality of said offset adapters having protrusions disposed at different angular positions relative to said first axis of said first tapered cylindrical portion and to said second axis of said second tapered cylindrical portion, and said plurality of said offset adapters being operable such that said central axis of said humeral head is differently eccentrically disposed relative to said central axis of said humeral anchor.

4. The humeral head implant system of claim 1, wherein an angular position of said protrusion relative to said first axis of said first tapered cylindrical portion and an angular position of said recess in said cavity relative to said first axis of said cavity being operable so that when said humeral head is assembled to said offset adapter said central axis of said humeral head is disposed concentrically with said central axis of said humeral anchor.

5. The humeral head implant system of claim 4, wherein said offset adapter and said humeral head comprise indicia associated with a relative amount of eccentricity between said humeral head and said second portion of said offset adapter when assembled.

6. The humeral head implant system of claim 1, wherein said humeral head comprises indicia for use in aligning said humeral head to a resected humerus.

7. The humeral head implant system of claim 1, wherein said humeral anchor comprises at least one of a stem or is stemless.

8. The humeral head implant system of claim 1, wherein the protrusion has a third axis and the third axis is offset from said first axis of said first tapered cylindrical portion of said offset adapter and from said second axis of said second tapered cylindrical portion.

9. The humeral head implant system of claim 8, wherein an angular position of said protrusion relative to said first axis of said first tapered cylindrical portion and to said second axis of said second tapered cylindrical portion is operable to provide a central axis of the humeral head to be disposed eccentrically relative to a central axis of the humeral anchor.

10. The humeral head implant system of claim 8, wherein said offset adapter comprises:
a plurality of said offset adapters, each of said plurality of said offset adapters having protrusions disposed at different angular positions relative to said first axis of said first tapered cylindrical portion and to said second axis of said second tapered cylindrical portion, and said plurality of said offset adapters being operable to provide a central axis of the humeral head to be differently eccentrically disposed relative to a central axis of the humeral anchor.

11. The humeral head implant system of claim 8, wherein an angular position of said protrusion relative to said first axis of said first tapered cylindrical portion and an angular position of said recess in said cavity relative to said first axis of said cavity is operable so that when the humeral head is assembled to said offset adapter a central axis of the humeral head it is disposed concentrically with a central axis of the humeral anchor.

12. The humeral head implant system of claim 8, wherein said offset adapter comprises indicia alignable with the humeral head for indicating an amount of eccentricity between the humeral head and said second portion of said offset adapter when assembled.
